(19) United States
(12) Patent Application Publication
(10) Pub. No.: US 2018/0049998 A1
(43) Pub. Date: Feb. 22, 2018

(54) Naphthoquinone Compounds and Methods for Preparing the Same

(71) Applicants: Erik S. Goebel, Saint Paul, MN (US); George Barany, Falcon Heights, MN (US)

(72) Inventors: Erik S. Goebel, Saint Paul, MN (US); George Barany, Falcon Heights, MN (US)

(21) Appl. No.: 15/681,332

(22) Filed: Aug. 18, 2017

Related U.S. Application Data
(60) Provisional application No. 62/376,609, filed on Aug. 18, 2016.

(51) Int. Cl. A61K 31/122; A61K 31/19; A61K 31/215; C07C 46/02; C07C 46/10; C07C 50/14; C07C 50/32

(52) U.S. Cl. CPC A61K 31/122; A61K 31/19; A61K 31/215; C07C 50/32; C07C 46/10; C07C 50/14; C07C 46/02

(57) ABSTRACT

The present application provides vitamin K metabolite derivatives and methods for preparing vitamin K metabolites and metabolite derivatives.

### RELATED APPLICATION

This application claims priority from, and the benefit of, U.S. Provisional Application No. 62/376,609, filed on Aug. 18, 2016, the entire contents of which are hereby incorporated by reference.

### BACKGROUND

The vitamin K family is divided into the plant-derived phylloquinone (vitamin K1), having a phytyl side-chain, and the menadiones (vitamin K2), which have isoprenoid side-chains of varying lengths. In humans, vitamin K is essential for blood coagulation and bone health (Vermeer, C.; Gijsbers, B. L. M. G.; Crackin, A. M.; Groenen-Van Dooren, M. C. L.; Knapen, M. H. J. J. Nutr. 1996, 126, 1187S), as well as further functions (Cockayne, S.; Adamson, J.; Lanham-New, S.; Shearer, M. J.; Gilbody, S.; Torgerson, D. J. Arch. Intern. Med. 2006, 166, 1256). Many biologically active vitamin K metabolites are known to promote blood clotting, promote osteoblast formation, inhibit osteoclast formation. (Mc Burney, A.; Shearer, M. J.; Barkhan P. Biochem. Med. 1980, 24, 250; Soper, R. J.; Oguz, C.; Emery, R.; Pitsillides, A. A.; Hodges, S. J. Mol. Nutr. Food Res. 2014, 58, 1658; and Shearer, M. J.; Bach, A.; Kohlmeier, M. J. Nutr. 1996, 126, 1181S). In order to study the physical and biological effects of vitamin K metabolites and their derivatives, efficient access to vitamin K metabolites and metabolite derivatives is necessary. Previously reported syntheses of vitamin K metabolites are costly and onerous (Fujii, S.; Shimizu, A.; Takeda, N.; Oguchi, K.; Katsurai, T. Shirakawa, H.; Komai, M.; Kagechika, H. Bioorg. Med. Chem. 2015, 23, 2344; Watanabe, M.; Okamoto, K.; Imada, I.; Morimoto, H. Chem. Pharm. Bull. 1978, 26, 774; Teitelbaum, A. M.; Scian, M.; Nelson, W. L.; Rettie, A. E. Synthesis 2015, 47, 944).

The present application appreciates that a facile, cost-effective, synthesis of vitamin K metabolites and metabolite derivatives may be a challenging endeavor.

### SUMMARY

In one embodiment, a compound represented by formula I is provided:

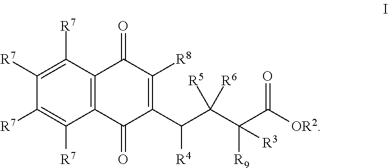

R² may include: —H, C₁-C₁₂ alkyl, C₆-C₁₀ aryl, or C₇-C₁₁ alkylene-aryl optionally substituted with one or more of: R^{a}, and OR^{b}. R^{a} and R^{b} may independently include: —H, C₁-C₆ alkyl, or —CF₃. R³ may include: —H, C₁-C₁₂ alkyl, or C₆-C₁₀ aryl. R⁴, R⁵ and R⁶ may independently include: —H, C₁-C₆ alkyl, or C₄-C₁₄ aryl or heteroaryl. Each R⁷ may independently include: —H, halogen, —NO₂, C₁-C₆ alkyl, —N₃, or C₄-C₁₄ aryl or heteroaryl. R⁸ may include: —H, halogen, —NO₂, C₁-C₆ alkyl, —N₃, or C₄-C₁₄ aryl or heteroaryl. R⁹ may include: —H or —C(O)OR². The compound may not be represented by formula 5:

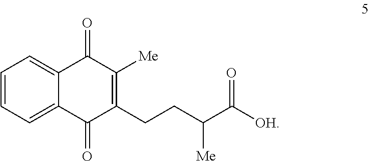 [SMILES: CC1=C(CCC(C)C(=O)O)C(=O)c2ccccc2C1=O]

In one embodiment, a method for preparing a compound represented by formula I is provided:

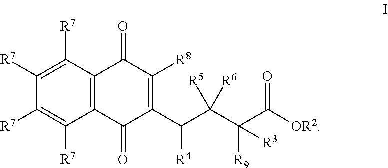

The method may include contacting an acrylate ester compound with a 1,3-diester compound in the presence of a base under a first set of conditions effective to form a triester compound. The method may include contacting a triester compound with a redox reagent under a second set of conditions effective to form a diester acid compound. The method may include contacting a diester acid compound with a naphthoquinone compound in the presence of a radical initiator under a third set of conditions effective to form a naphthoquinone diester compound. The method may include contacting a naphthoquinone diester compound with a hydrolysis reagent or a saponification reagent to form a solution under a fourth set of conditions effective to form the compound of formula I. The acrylate ester compound, the 1,3-diester compound, the triester compound, the diester acid compound, the naphthoquinone compound, and the naphthoquinone diester compound may be represented by formulas A-F, respectively:

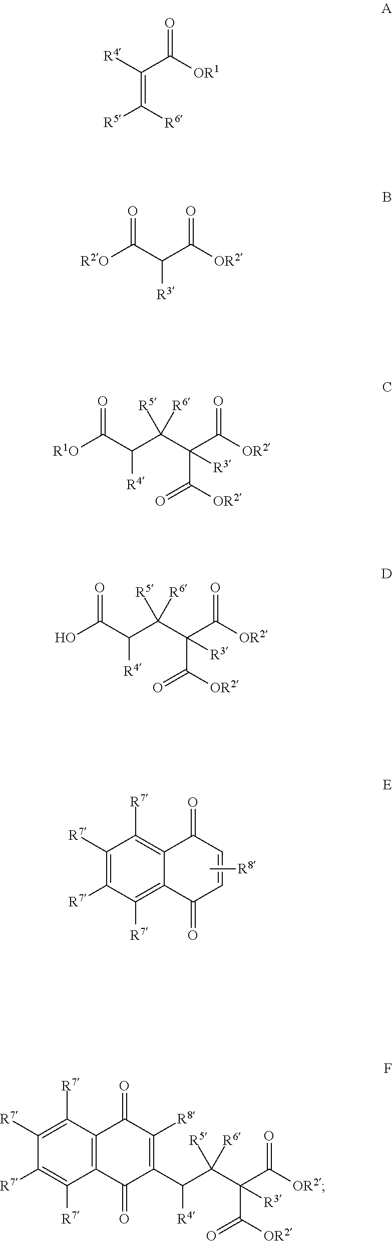

R¹ may include: C₇-C₁₁ alkylene-aryl optionally substituted with one or more of: R^{a}, and OR^{b}. R^{a} and R^{b} may independently include: —H, C₁-C₆ alkyl, or CF₃. Each R² may independently include: —H, C₁-C₁₂ alkyl, C₆-C₁₀ aryl, or C₇-C₁₁ alkylene-aryl optionally substituted with one or more of: R^{a}′, and OR^{b}′. Each R²′ may independently include: C₁-C₁₂ alkyl, C₆-C₁₀ aryl, or C₇-C₁₁ alkylene-aryl optionally substituted with one or more of: R^{a}″, and OR^{b}″. R^{a}″ and R^{b}″ may independently include: —H, C₁-C₆ alkyl, or CF₃. R³ and R³′ may include: —H, C₁-C₁₂ alkyl, or C₆-C₁₀ aryl. If R²′ is C₇ alkylene-aryl, then R¹ may include C₁₁ alkylene-aryl, or C₇-C₁₁ alkylene-aryl substituted with OR^{b}, wherein R^{b} is C₁-C₆ alkyl. Alternatively, if R²′ is C₇ alkylene-aryl substituted with one or more of: R^{a}′, and OR^{b}′ wherein R^{a}′ and R^{b}′ are independently —H, C₁-C₆ alkyl, or CF₃, then R¹ may include C₇-C₁₁ alkylene-aryl. R⁴, R⁴′, R⁵, R⁵′, R⁶, and R⁶′ are independently —H, C₁-C₆ alkyl, or C₄-C₁₄ aryl or heteroaryl. Each R⁷ each R⁷′, R⁸, and R⁸′ may independently include; —H, halogen, —NO₂, C₁-C₆ alkyl, —N₃, or C₄-C₁₄ aryl or heteroaryl. R⁹ may be include: —H or —C(O)OR².

### DETAILED DESCRIPTION

The present application relates to vitamin K metabolites and derivatives thereof having utility as LC/MS standards to measure levels of vitamin K in mammals, as blood clotting factors, and as osteoblastogenesis promoters and osteoclastogenesis inhibitors. In particular, the present application is directed toward vitamin K metabolite compounds, metabolite-like compounds, and methods for preparing the same. The methods provided herein allow for cost-effective syntheses of a vitamin K human metabolite, e.g., compound 5, as well as convenient routes to various metabolite derivatives. Further, the methods provided herein avoid the use of protecting groups, avoid the need to exclude air, and allow for a single purification step.

In various embodiments, a compound represented by formula I is provided:

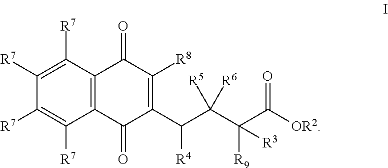

R² may include: —H, C₁-C₁₂ alkyl, C₁-C₁₂ alkyl optionally substituted with one or more of the following: —CO(O)H, —OH, C₆-C₁₀ aryl, —CN, —COS, —NH₂, —NH₂(NH)NH₃, —OSO₃H, —SO₃H, —SH, or C₆-C₁₀ aryl, or C₇-C₁₁ alkylene-aryl optionally substituted with one or more of: R^{a}, and OR^{b}. R^{a} and R^{b} may independently include: —H, C₁-C₆ alkyl, or —CF₃. R³ may include: —H, C₁-C₁₂ alkyl, C₆-C₁₀ aryl, or C₇-C₁₁ alkylene-aryl. R⁴, R⁵ and R⁶ may independently include: —H, C₁-C₆ alkyl, or C₄-C₁₄ aryl or heteroaryl. Each R⁷ may independently include: —H, halogen, —NO₂, C₁-C₆ alkyl, —N₃, or C₄-C₁₄ aryl or heteroaryl. R⁸ may include: —H, halogen, —NO₂, C₁-C₆ alkyl, —N₃, or C₄-C₁₄ aryl or heteroaryl. R⁹ may include: —H or —C(O)OR². The compound may not be represented by formula IA:

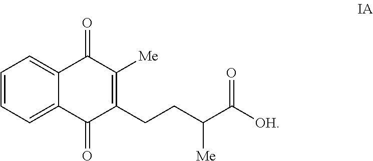 [SMILES: CC1=C(CCC(C)C(=O)O)C(=O)c2ccccc2C1=O]

In many embodiments, R³ may include a linear or branched C₁-C₁₂ alkyl. R³ may include C₁-C₄ alkyl. For example, R³ may include —CH₃, —CH₂CH₃, —CH₂CH₂CH₃, —CH₂CH₂CH₂CH₃, —CH(CH₃)₂, —CH₂CH₂(CH₃)₂, —C(CH₃)₃, or the like. For example, R³ may include —CH₃. In other embodiments, R³ may include C₆-C₁₀ aryl. For example, R³ may include phenyl. In some embodiments, R³ may include —H.

In many embodiments, R⁴ may include a linear or branched C₁-C₁₂ alkyl. R⁴ may include C₁-C₄ alkyl. For example, R⁴ may include —CH₃, —CH₂CH₃, —CH₂CH₂CH₃, —CH₂CH₂CH₂CH₃, —CH(CH₃)₂, —CH₂CH₂(CH₃)₂, —C(CH₃)₃, or the like. For example, R⁴ may include —CH₃. In other embodiments, R⁴ may include C₆-C₁₀ aryl. For example, R⁴ may include phenyl or naphthyl. In some embodiments, R⁴ may include —H.

In many embodiments, R⁴ may include a linear or branched C₁-C₁₂ alkyl. R⁴ may include C₁-C₄ alkyl. For example, R⁴ may include —CH₃, —CH₂CH₃, —CH₂CH₂CH₃, —CH₂CH₂CH₂CH₃, —CH(CH₃)₂, —CH₂CH₂(CH₃)₂, —C(CH₃)₃, or the like. For example, R⁴ may include —CH₃. In other embodiments, R⁴ may include C₆-C₁₀ aryl. For example, R⁴ may include phenyl or naphthyl. In some embodiments, R⁴ may include —H.

In many embodiments, R⁵ may include a linear or branched C₁-C₁₂ alkyl. R⁵ may include C₁-C₄ alkyl. For example, R⁵ may include —CH₃, —CH₂CH₃, —CH₂CH₂CH₃, —CH₂CH₂CH₂CH₃, —CH(CH₃)₂, —CH₂CH₂(CH₃)₂, —C(CH₃)₃, or the like. For example, R⁵ may include —CH₃. In other embodiments, R⁵ may include C₆-C₁₀ aryl. For example, R⁵ may include phenyl or naphthyl. In some embodiments, R⁵ may include —H.

In many embodiments, R⁶ may include a linear or branched C₁-C₁₂ alkyl. R⁶ may include C₁-C₄ alkyl. For example, R⁶ may include —CH₃, —CH₂CH₃, —CH₂CH₂CH₃, —CH₂CH₂CH₂CH₃, —CH(CH₃)₂, —CH₂CH₂(CH₃)₂, —C(CH₃)₃, or the like. For example, R⁶ may include —CH₃. In other embodiments, R⁶ may include C₆-C₁₀ aryl. For example, R⁶ may include phenyl or naphthyl. In some embodiments, R⁶ may include —H.

In other embodiments, R⁵ may be taken together with R⁶ to form a C₃-C₆ cycloalkyl. R⁵ and R⁶ may be taken together to form, for example, a cyclopropyl, cyclobutyl, cyclopentyl, or cyclohexyl group.

In many embodiments, R⁷ may, independently together, include a linear or branched C₁-C₁₂ alkyl. R⁷ may include C₁-C₄ alkyl. For example, R⁷ may include —CH₃, —CH₂CH₃, —CH₂CH₂CH₃, —CH₂CH₂CH₂CH₃, —CH(CH₃)₂, —CH₂CH₂(CH₃)₂, —C(CH₃)₃, or the like. For example, R⁷ may include —CH₃. In other embodiments, R⁷ may include C₆-C₁₀ aryl. For example, R⁷ may include phenyl or naphthyl. Each R⁷ may independently include H, C₁-C₆ alkyl, C₆-C₁₀ aryl, halogen, —NO₂, —C(O)R′, —C(O)OR′, —OC(O)R′, —OR′, —OC(O)N(R′)₂, —N(R′)C(O)R′, —N(R′)₂, —N(R′)C(O)OR′, —N(R′)(NR′)N(R′)₂, C₁-C₆ alkyl substituted with one or more: OR′, halogen, or —N(R′)₂; wherein R′ is H or C₁-C₆ alkyl.

In many embodiments, R⁸ may include a linear or branched C₁-C₁₂ alkyl. R⁸ may include C₁-C₄ alkyl. For example, R⁸ may include —CH₃, —CH₂CH₃, —CH₂CH₂CH₃, —CH₂CH₂CH₂CH₃, —CH(CH₃)₂, —CH₂CH₂(CH₃)₂, —C(CH₃)₃, or the like. For example, R⁸ may include —CH₃. In other embodiments, R⁸ may include C₆-C₁₀ aryl. For example, R⁸ may include phenyl or napthyl. In some embodiments, R⁸ may include —H. R⁸ may include H, C₁-C₆ alkyl, C₆-C₁₀ aryl, halogen, —NO₂, —C(O)R′, —C(O)OR′, —OC(O)R′, —OR′, —OC(O)N(R′)₂, —N(R′)C(O)R′, —N(R′)₂, —N(R′)C(O)OR′, —N(R′)(NR′)N(R′)₂, C₁-C₆ alkyl substituted with one or more: OR′, halogen, or —N(R′)₂; wherein R′ is H or C₁-C₆ alkyl.

All combinations disclosed herein have been contemplated.

In several embodiments R⁹ may include —C(O)OR². In other embodiments, R⁹ may include —H. R⁹ may include —C(O)OR² and R² may include —H. R⁹ may include —C(O)OR² and R² may include —CH₂CH₃. In many embodiments, R⁹ may be —H and R² may be —H.

In various embodiments, a method for preparing a compound represented by formula I is provided:

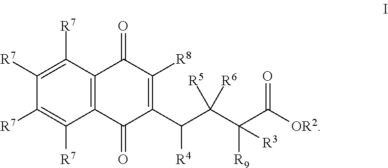

The method may include contacting an acrylate ester compound A with a 1,3-diester compound B in the presence of a base under a first set of conditions effective to form a triester compound C. The method may include contacting a triester compound C with a redox reagent under a second set of conditions effective to form a diester acid compound D. The method may include contacting a diester acid compound D with a naphthoquinone compound E in the presence of a radical initiator under a third set of conditions effective to form a naphthoquinone diester compound F. The method may include contacting a naphthoquinone diester compound F with a hydrolysis reagent, saponification reagent, or halide salt reagent to form a solution under a fourth set of conditions effective to form the compound of formula I. The acrylate ester compound, the 1,3-diester compound, the triester compound, the diester acid compound, the naphthoquinone compound, and the naphthoquinone diester compound may be represented by formulas A-F, respectively:

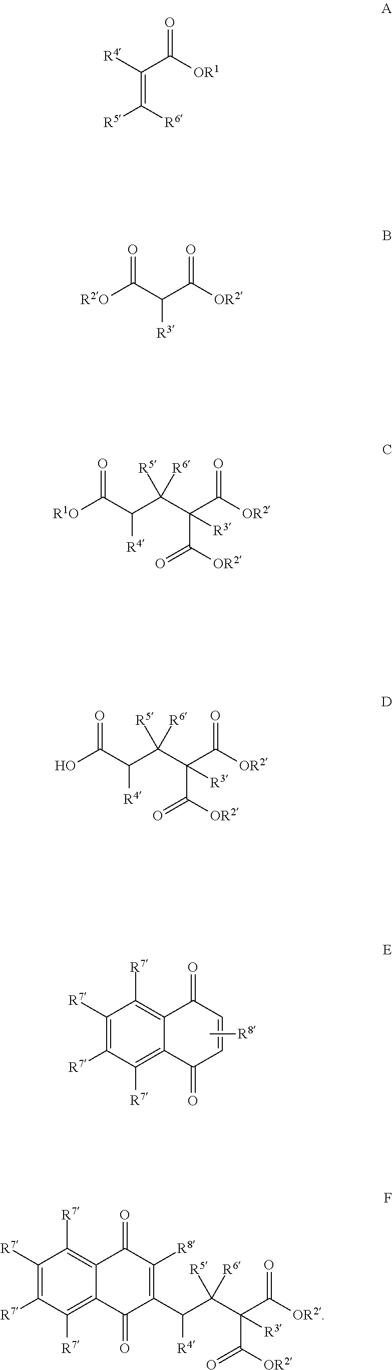

In some embodiments, compound I may be represent by formulas IB or IC:

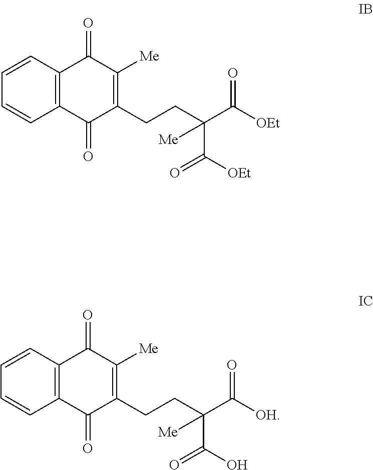 [SMILES: CCOC(=O)C(C)(CCC1=C(C)C(=O)c2ccccc2C1=O)C(=O)OCC ; CC1=C(CCC(C)(C(=O)O)C(=O)O)C(=O)c2ccccc2C1=O]

R¹ may include: C₇-C₁₁ alkylene-aryl optionally substituted with one or more of: R^{a}, and OR^{b}. R^{a} and R^{b} may independently include: —H, C₁-C₆ alkyl, or CF₃. Each R² may independently include: —H, C₁-C₁₂ alkyl, C₆-C₁₀ aryl, or C₇-C₁₁ alkylene-aryl optionally substituted with one or more of: R^{a}′, and OR^{b}′. Each R²′ may independently include: C₁-C₁₂ alkyl, C₆-C₁₀ aryl, or C₇-C₁₁ alkylene-aryl optionally substituted with one or more of: R^{a}″, and OR^{b}″. R^{a}″ and R^{b}″ may independently include: —H, C₁-C₆ alkyl, or CF₃. R³ and R³′ may include: —H, C₁-C₁₂ alkyl, or C₆-C₁₀ aryl. If R²′ is C₇ alkylene-aryl, then R¹ may include C₁₁ alkylene-aryl, or C₇-C₁₁ alkylene-aryl substituted with OR^{b}, wherein R^{b} is C₁-C₆ alkyl. Alternatively, if R²′ is C₇ alkylene-aryl substituted with one or more of: R^{a}′, and OR^{b}′ wherein R^{a}′ and R^{b}′ are independently —H, C₁-C₆ alkyl, or CF₃, then R¹ may include C₇-C₁₁ alkylene-aryl. R⁴, R⁴′, R⁵, R⁵′, R⁶, and R⁶′ are independently —H, C₁-C₆ alkyl, or C₄-C₁₄ aryl or heteroaryl. Each R⁷, each R⁷′, R⁸, and R⁸′ may independently include; —H, halogen, —NO₂, C₁-C₆ alkyl, —N₃, or C₄-C₁₄ aryl or heteroaryl. R⁹ may be include: —H or —C(O)OR².

In many embodiments, the acrylate ester compound may be contacted with the 1,3-diester compound in the presence of a base. The base may include a hydroxide, carbonate, or hydride salt. For example, the base may include sodium hydroxide, potassium hydroxide, sodium carbonate, potassium carbonate, cesium carbonate, sodium hydride, potassium hydride, or the like. In several embodiments, the base may include sodium hydroxide. In other embodiments, the base may include an amine or amine anion salt. For example, the base may include trimethylamine, triethylamine, N,N-diisopropylethylamine (Hünigs base), lithium diisopropylamide (LDA), pyridine, or the like. In some embodiments, the corresponding conjugate acid of the base may be characterized as having a pKa greater than about 15 in water or greater than about 30 in DMSO. For example, the pKa in water may be greater than one or more of: 5, 10, 15, 20, 25, 30, and 35.

In some embodiments, the first set of conditions may include the substantial absence of a solvent. For example, the acrylate ester compound may be contacted with the 1,3-diester compound without solvent, e.g., ethyl acetate, ethanol, methanol, water, and the like. The first set of conditions may be substantially solventless and the base may include a hydroxide salt. For example, the acrylate ester compound may be contacted with the 1,3-diester compound neat in the presence of hydroxide salt, e.g., sodium hydroxide. The substantial absence of solvent may refer to an amount of solvent in (w/w %) with respect to the remaining components, e.g., acrylate ester, 1,3-diester, base, of less than about one or more of: 1, 0.9, 0.8, 0.7, 0.6, 0.5, 0.4, 0.3, 0.2, 0.1, 0.05, 0.02, 0.005, and 0.001. The substantial absence of solvent may refer to a range in (w/w %) between any of the preceding values, for example, between about 0.5 and about 0.2, between about 0.001 and about 0.02, or the like. The substantial absence of solvent may refer to amount of solvent in (w/w %) of less than about 0.001.

In some embodiments, the first set of conditions may include allowing the acrylate ester compound and the diester compound to react in the presence of base for a period of time in minutes of less than about one or more of: 15, 14, 13, 12, 11, 10, 9, 8, 7, 6, 5, 4, 3, 2, 1, and 0.5. The period of time may be in a range between any of the preceding values, for example, between about 1 and 3 minutes, between about 7 and 10 minutes, or the like. The acrylate ester compound and the diester compound may be allowed react for a period of time of about 5 minutes. The preceding time values may refer to the acrylate ester being present in an amount in mmol of about one or more of: 5, 10, 20, 30, 40, 50, 60, 70, 80, 90, 100, 110, 120, 130, 140, 150, 160, 170, 180, 190, 200, 250, 300, 350, 400, 350, and 500, or between any of the preceding values, for example, between about 100 and about 130. For example the contacting of about 117 mmol of the acrylate ester compound with the diester compound may be allowed to for about 5 minutes. The time required to react may be longer on larger scale, e.g., greater than about 1 kg)

In some embodiments, the base may be removed prior to contacting the triester with the redox reagent. For example, the base may be removed via filtration. For example, the base may include sodium hydroxide and the sodium hydroxide may be removed via filtration. In other embodiments, the base may be removed via aqueous workup and extraction with an organic solvent, e.g., ethyl acetate, diethylether, or the like.

In several embodiments, the triester compound may be contacted with a redox reagent to form the diester acid compound. The redox reagent may include one or more of: a catalyst, H₂, a hydride reagent, and a single-electron transfer (SET) oxidant. For example, the redox reagent may include a catalyst and H₂. The catalyst may include, for example, one or more of: palladium (Pd), platinum (Pt), rhodium (Rh), iridium (Ir), ruthenium (Ru), Nickel (Ni), and the like. The catalyst may include palladium on carbon (Pd/C), for example. The catalyst may include palladium tetrakis (Pd(PPh₃)₄). The catalyst may include a pre-catalyst, e.g., palladium acetate, and a reducing ligand, e.g., a phosphine ligand, e.g., triphenylphosphine. In other embodiments, the redox reagent may include a single-electron transfer oxidant. For example, the SET oxidant may include dichlorodicyanobenzoquinone (DDQ).

In several embodiments, the diester acid compound may be contacted with a naphthoquinone compound in the presence of a radical initiator. The radical initiator may include a peroxydisulfate salt. The peroxydisulfate salt may include, for example ammonium persulfate, potassium persulfate, or the like. The radical initiator may include a silver(I) salt. For example, the silver(I) salt may include silver nitrite, silver nitrate, silver carbonate, silver chloride, silver chromate, silver citrate, silver cyanate, silver fluoride, silver heptafluorobutyrate, or the like. In many embodiments the radical initiator may include a peroxydisulfate salt and silver (I) salt. For example, the radical initiator may include ammonium persulfate and silver nitrite.

In several embodiments, the naphthoquinone diester compound may be contacted with a hydrolysis reagent. The hydrolysis reagent may include a Brønstad acid. For example, the Brønstad acid may include hydrochloric acid, hydrobromic acid, hydroiodic acid, p-toluene sulfonic acid, trifluoroacetic acid, acetic acid, or the like.

In other embodiments, the naphthoquinone diester compound may be contacted with a saponification reagent. The saponification reagent may include, for example, a Brønstad base. The saponification may include water. The saponification may include a Brønstad base, e.g., a hydroxide salt, e.g., sodium hydroxide, and water.

In other embodiments, the naphthoquinone diester compound may be contacted with a halide salt reagent. The halide salt reagent may include any salt including Br⁻, I⁻, or Cl⁻, or F⁻. For example the halide salt reagent may include LiCl, LiBr, KCl, KBr, KI, NaCl, NaBr, NaI, KF, NH₄Cl, and the like.

In many embodiments, the fourth set of conditions may include heating the solution to a temperature in ° C. of about one or more of: 30, 40, 50, 60, 70, 80, 90, 100, 110, 120, 130, 140, and 150. The solution may be heated to a temperature in ° C. in a range between any of the preceding values, for example, between about 90 and about 100, between about 100 and about 120, and the like. The solution may be heated to a temperature of at least 100° C.

In many embodiments, the fourth set of conditions may include heating the solution for a period of time in minutes of about one or more of: 5, 10, 20, 30, 40, 50, 60, 90, 120, 150, 160, 170, 180, 190, 200, 210, 240, 270, 300, 360, 420, 480, 540, and 600. The solution may be heated for a period of time in a range between any of the preceding values, for example, between about 60 and about 270, between about 150 and about 210, and the like. For example, the solution may be heated to at least about 100° C. for about 160 to about 210 minutes. The solution may be heated at a temperature for a period of time greater than 600 minutes.

In some embodiments, the naphthoquinone diester compound may be contacted with the hydrolysis reagent and the solution may be heated to at least about 100° C.

In many embodiments, the fourth set of conditions may include heating the solution at a temperature for a period of time and subsequently contacting the solution with a decarboxylation promoter. A decarboxylation promoter may include, for example, N,N-carbonyldiimidazole (CDI), phosgene, trifluoroacetic anhydride, or the like. In some embodiments, the decarboxylation promoter may include N,N-carbonyldiimidazole (CDI).

In some embodiments, the naphthoquinone diester compound may be contacted with the hydrolysis reagent and the solution may be heated to at least about 100° C. for any period of time previously disclosed. The solution may be subsequently contacted with a decarboxylation promoter.

### EXAMPLES

Starting materials and solvents were used as received from commercial sources. Melting points were taken on a Fisher-Johns melting point apparatus and are uncorrected. NMR spectra were obtained on either a 500 MHz Bruker Avance III or a 400 MHz Bruker Avance III HD and all spectra are referenced to CDCl₃. IR spectra were obtained on NaCl plates, using a Thermo Nicolet iS 5 FT-IR spectrometer. HRMS was obtained on a Bruker BioTOF II with PEG internal standard. Column chromatography was accomplished with SiliaFlash P60 silica gel.

### Example 1: 5-ethoxy-4-(ethoxycarbonyl)-4-methyl-5-oxopepntanoic Acid

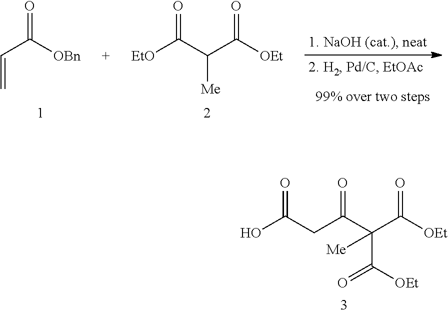 [SMILES: C=CC(=O)OCc1ccccc1 ; CCOC(=O)C(C)C(=O)OCC ; CCOC(=O)C(C)(C(=O)CC(=O)O)C(=O)OCC]

Sodium hydroxide pellets (470 mg, 11.7 mmol) were freshly crushed in a mortar and pestle, and added to a stirred mixture of benzyl acrylate (1) (17.5 mL, 117 mmol) and diethyl methylmalonate (2) (20.0 mL, 117 mmol) with an external room temperature (about 25° C.) water bath. After about 5 minutes (min), the mixture was diluted with ethyl acetate (250 mL) and filtered through a plug of silica. 10% Pd/C was added (624 mg, 0.60 mol) to the crude 1-benzyl-3,3-diethylbutane-1,3,3-tricarboxylate as a solution in ethyl acetate (250 mL). A balloon containing H₂ was attached to the flask, and the reaction was allowed to stir overnight. The mixture was filtered and the filtrate was concentrated to provide spectroscopically pure 5-ethoxy-4-(ethoxycarbonyl)-4-methyl-5-oxopepntanoic acid (3) as a light tan oil (28.5 g, 99%). ¹H NMR (400 MHz, CDCl₃) δ 4.19 (q, J=7.1 Hz, 4H), 2.38-2.49 (m, 2H), 2.15-2.25 (m, 2H), 1.43 (s, 3H), 1.26 (t, J=7.1 Hz, 6H); ¹³C NMR (100 MHz, CDCl₃) δ 178.7, 171.7, 61.4, 52.8, 30.3, 29.5, 29.4, 20.2, 14.0; IR (neat): 2985, 2942, 1731, 1299, 1264, 1243, 1110, 1022, 861 cm⁻¹; HRMS (ESI): m/z calcd for C₁₁H₁₈O₆Na⁺ [M+Na]⁺: 269.0996; found: 269.1004.

### Example 2: diethyl-2-methyl-2-(2-(3-methyl-1,4-dioxo-1,4-dihydronaphthalen-2-yl)ethyl)malonate

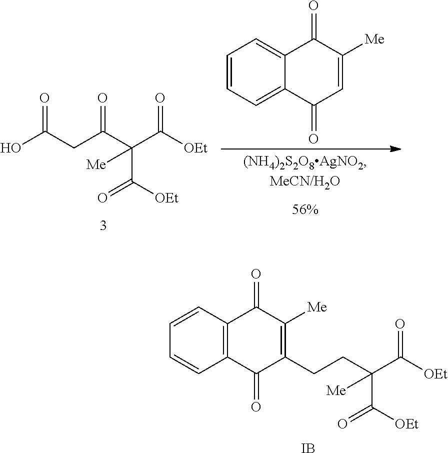 [SMILES: CCOC(=O)C(C)(C(=O)CC(=O)O)C(=O)OCC ; CC1=CC(=O)c2ccccc2C1=O ; CCOC(=O)C(C)(CCC1=C(C)C(=O)c2ccccc2C1=O)C(=O)OCC ; "IB"]

A solution of 5-ethoxy-4-(ethoxycarbonyl)-4-methyl-5-oxopepntanoic acid (3) (8.38 g, 34.1 mmol), 2-methylnaphthalene-1,4-dione (2.00 g, 11.4 mmol), and AgNO₃ (580 mg, 3.41 mmol) in MeCN (acetonitrile)/H₂O (2:1; 84 mL) was warmed to about 75° C., and a solution of ammonium persulfate (3.37 g, 14.8 mmol) in MeCN/H₂O (1:1; 56 mL) was added dropwise over 1 hour. After a further about 20 hours of stirring at about 75° C., the reaction mixture was cooled, diluted with H₂O (250 mL), and a saturated aqueous solution of NaHCO₃ (250 mL) was added. Ethyl acetate (EtOAc) (500 mL+250 mL) was used to extract the product and the combined organic layers were washed with brine (100 mL), dried over (Na₂SO₄), concentrated, and maintained overnight at room temperature. The crystalline solid that formed was triturated with MeOH (methanol)/H₂O (9:1; 50 mL). Diethyl-2-methyl-2-(2-(3-methyl-1,4-dioxo-1,4-dihydronaphthalen-2-yl)ethyl)malonate (IB) was collected as a yellow, free-flowing powder. Yield: 2.27 g (53%). mp: 96° C. ¹H NMR (400 MHz, CDCl₃) δ 8.08 (dd, J=5.6, 3.2 Hz, 1H) 7.70 (dd, J=5.6, 3.2 Hz, 2H) 4.24 (q, J=7.1 Hz, 4H) 2.57-2.68 (m, 2H) 2.22 (s, 3H) 1.92-2.03 (m, 2H) 1.57 (s, 3H) 1.30 (t, J=7.1 Hz, 6H); ¹³C NMR (125 MHz, CDCl₃) δ 185.1, 184.2, 171.9, 146.1, 143.9, 133.3, 132.1, 126.2 (×2), 61.4, 53.6, 34.0, 22.2, 19.7, 14.1, 12.4; IR (neat): 2992, 2940, 1730, 1660, 1296, 1181, 1110, 1020, 718 cm⁻¹; HRMS (ESI): m/z calcd for C₂₁H₂₄O₆Na⁺ [M+Na]⁺: 395.1465; found: 395.1478.

### Example 3: 2-methyl-4-(3-methyl-1,4-dioxo-1,4-dihydronaphthalen-2-yl)butanoic Acid

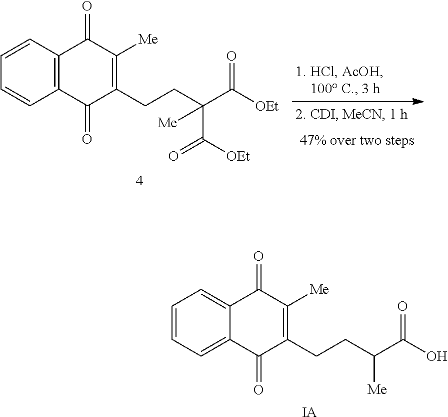

A solution of diethyl-2-methyl-2-(2-(3-methyl-1,4-dioxo-1,4-dihydronaphthalen-2-yl)ethyl)malonate (IB) (1.79 g, 4.8 mmol) in AcOH (acetic acid)/conc. HCl/water (2:1:1; 96 mL) was heated to about 100° C. for about 3 hours and cooled to room temperature. EtOAc (100 mL) was added, followed by washes with H₂O (2×100 mL). The organic layer was dried (Na₂SO₄) and concentrated to give a brown oil which was dissolved in MeCN (100 mL) and reacted with N,N′-carbonyldiimidazole (1.02 g, 6.31 mmol) for about 1 h with stirring. 1 M aqueous NaOH (50 mL) was added, the mixture was stirred an additional 15 min, and the reaction was acidified with aqueous HCl (1M, 100 mL) and extracted into EtOAc (2×250 mL). The combined organic phases were washed with H₂O (2×100 mL), brine (100 mL), dried (Na₂SO₄), and concentrated. Column chromatography (30% EtOAc/hexanes with 1% AcOH) gave pure 2-methyl-3-(3′,3′-carboxymethylpropyl)-1,4-naphthoquinone (1A) as a yellow solid after concentration. Yield: 608 mg (47%); mp 108-110° C. [lit. mp 112-113° C.]; ¹H NMR (500 MHz, CDCl₃): δ 8.09 (dt, J=5.9, 2.7 Hz, 2H), 7.68-7.74 (m, 2H), 2.58-2.79 (m, 3H), 2.22 (s, 3H), 1.80-1.94 (m, 1H), 1.59-1.72 (m, 1H), 1.31 (d, J=7.0 Hz, 3H); ¹³CNMR (125 MHz, CDCl₃) δ 185.2, 184.5, 182.2, 146.3, 143.8, 133.4, 132.1, 126.2, 39.5, 31.8, 24.8, 17.0, 12.5; IR (neat): 2975, 2939, 1705, 1659, 1461, 1378, 1296, 1182, 714, 692 cm⁻¹; HRMS (ESI): m/z calcd for C₁₈H₂₀O₄Na⁺ [M+Na]⁺: 295.0941; found: 295.0914.

As used herein, an “alkyl” group includes straight chain and branched chain alkyl groups having a number of carbon atoms, for example, from 1 to 12, 1 to 10, 1 to 8, 1 to 6, or 1 to 4. Examples of straight chain alkyl groups include groups such as methyl, ethyl, n-propyl, n-butyl, n-pentyl, n-hexyl, n-heptyl, and n-octyl groups. Examples of branched alkyl groups include, e.g., isopropyl, iso-butyl, sec-butyl, tert-butyl, neopentyl, isopentyl, and 2,2-dimethylpropyl groups. Representative substituted alkyl groups may be substituted one or more times with substituents such as those listed above and include, without limitation, haloalkyl (e.g., trifluoromethyl), hydroxyalkyl, thioalkyl, aminoalkyl, alkylaminoalkyl, dialkylaminoalkyl, alkoxyalkyl, or carboxyalkyl.

As used herein, an “alkoxy” group means a hydroxyl group (—OH) in which the bond to the hydrogen atom is replaced by a bond to a carbon atom of a substituted or unsubstituted alkyl group. Examples of linear alkoxy groups include, e.g., methoxy, ethoxy, propoxy, butoxy, pentoxy, or hexoxy. Examples of branched alkoxy groups include, e.g., isopropoxy, sec-butoxy, tert-butoxy, isopentoxy, or isohexoxy. Examples of cycloalkoxy groups include, e.g., cyclopropyloxy, cyclobutyloxy, cyclopentyloxy, or cyclohexyloxy. Representative substituted alkoxy groups may be substituted one or more times.

As used herein, a “cycloalkyl” group includes mono-, bi- or tricyclic alkyl groups having from 3 to 12 carbon atoms in each ring, for example, 3 to 10, 3 to 8, or 3 to 4, 5, or 6 carbon atoms. Exemplary monocyclic cycloalkyl groups include, for example, cyclopropyl, cyclobutyl, cyclopentyl, cyclohexyl, cycloheptyl, cyclooctyl, and the like. A cycloalkyl group may have a number of ring carbons of from 3 to 8, 3 to 7, 3 to 6, or 3 to 5. Bi- and tricyclic ring systems may include both bridged cycloalkyl groups and fused rings, e.g., bicyclo[2.1.1]hexane, adamantyl, decalinyl, and the like. Substituted cycloalkyl groups may be substituted one or more times with non-hydrogen and non-carbon groups as defined above. Substituted cycloalkyl groups may include rings that may be substituted with straight or branched chain alkyl groups. Representative substituted cycloalkyl groups may be mono-substituted or substituted more than once, for example, 2,2-, 2,3-, 2,4- 2,5- or 2,6-disubstituted cyclohexyl groups.

As used herein, an “aryl” group means a carbocyclic aromatic hydrocarbon. Aryl groups herein include monocyclic, bicyclic and tricyclic ring systems. Aryl groups include, e.g., phenyl, azulenyl, heptalenyl, biphenyl, fluorenyl, phenanthrenyl, anthracenyl, indenyl, indanyl, pentalenyl, naphthyl, and the like, for example, phenyl, biphenyl, and naphthyl. Aryl groups may contain, for example, 6 to 14, 6 to 12, or 6 to 10 ring carbons. In some embodiments, the aryl groups may be phenyl or naphthyl. Although the phrase “aryl groups” may include groups containing fused rings, such as fused aromatic-aliphatic ring systems (e.g., indanyl or tetrahydronaphthyl), an “aryl” group, unless stated to be substituted or optionally substituted, does not include aryl groups that have other groups, such as alkyl or halo groups, bonded to one of the ring members. Rather, groups such as tolyl may be referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl, which may be substituted with substituents such as those above.

As used herein, an “alkylene-aryl” group means an alkyl group in which a hydrogen or carbon bond of an alkyl group is replaced with a bond to an aryl group. In some embodiments, alkylene-aryl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative alkylene-aryl groups include, e.g., benzyl, i.e., —CH₂Ph, phenethyl groups, i.e, —CH₂CH₂Ph, and naphthylmethyl groups, i.e., —CH₂Naphth. Substituted alkylene-aryls may be substituted one or more times.

As used herein, a “heteroaryl” group means a carbocyclic aromatic ring having one or more ring carbon atoms replaced by a heteroatom (e.g., N, S, or O). Heteroaryl groups may include, for example, imidazolyl, isoimidazolyl, thienyl, furanyl, pyridyl, pyrimidyl, pyranyl, pyrazolyl, pyrrolyl, pyrazinyl, thiazoyl, isothiazolyl, oxazolyl, isooxazolyl, 1,2,3-trizaolyl, 1,2,4-triazolyl, and tetrazolyl. Heteroaryl groups also include fused polycyclic aromatic ring systems in which a carbocyclic aromatic ring or heteroaryl ring is fused to one or more other heteroaryl rings. Examples of heteroaryl groups may include benzothienyl, benzofuranyl, indolyl, quinolinyl, benzothiazolyl, benzoisothiazolyl, benzooxazolyl, benzoisooxazolyl, benzimidazolyl, quinolinyl, isoquinolinyl and isoindolyl.

Groups described herein having two or more points of attachment (e.g., divalent, trivalent, or polyvalent) within the compound of the technology may be designated by use of the suffix, “ene.” For example, divalent alkyl groups may be alkylene groups, divalent aryl groups may be arylene groups, divalent heteroaryl groups may be heteroarylene groups, and so forth.

As used herein, “optionally substituted” means a compound or group that may be substituted or unsubstituted. The term “substituted” refers to an organic group (e.g., an alkyl group) in which one or more bonds to a hydrogen atom contained therein may be replaced by a bond to non-hydrogen or non-carbon atoms. Substituted groups also include groups in which one or more bonds to a carbon or hydrogen atom may be replaced by one or more bonds, including double or triple bonds, to a heteroatom. A substituted group may be substituted with one or more substituents, unless otherwise specified. In some embodiments, a substituted group may be substituted with 1, 2, 3, 4, 5, or 6 substituents.

Examples of substituent groups include: halogens (F, Cl, Br, and I); hydroxyl; alkoxy, alkenoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; or nitriles.

In particular, suitable substituents for an alkyl group, cycloalkyl group, or an aryl group ring carbon are those which do not substantially interfere with the activity of the disclosed compounds. Examples include —OH, halogen (—Br, -, —I and —F), —OR^{A}, —O(CO)R^{A}, —(CO)R^{A}, —CN, —NO₂, —CO₂H, —SO₃H, —NH₂, —NHR^{A}, —N(R^{A}R^{B}), —(CO)OR^{A}, —(CO)H, —CONH₂, —CONHR^{A}, —CON(R^{A}R^{B}), —NHCOR^{A}, —NRCOR^{A}, —NHCONH₂, —NHCONR^{A}H, —NHCON(R^{A}R^{B}), —NR^{C}CONH₂, —NR^{C}CONR^{A}H, —NR^{C}CON(R^{A}R^{B}), —C(═NH)—NH₂, —C(═NH)—NHR^{A}, —C(═NH)—N(R^{A}R^{B}), —C(═NR^{C})—NH₂, —C(═NR^{C})—NHR^{A}, —C(═NR^{C})—N(R^{A}R^{B}), —NH—C(═NH)—NH₂, —NH—C(═NH)—NHR^{A}, —NH—C(═NH)—N(R^{A}R^{B}), —NH—C(═NR^{C})—NH₂, —NH—C(═NR^{C})—NHR^{A}, —NH—C(═NR^{C})—N(R^{A}R^{B}), NR^{D}H—C(═NH)—NH₂, —NR^{D}—C(═NH)—NHR^{A}, —NR^{D}—C(═NH)—N(R^{A}R^{B}), —NR^{D}—C(═NR^{C})NH₂, —NR^{D}—C(═NR^{C})—NHR^{A}, —NR^{D}—C(═NR^{C})—N(R^{A}R^{B}), —NHNH₂, —NHNHR^{A}, —NHR^{A}R^{B}, —SO₂NH₂, —SO₂NHR^{A}, —SO₂NR^{A}R^{B}, —CH═CHR^{A}, —CH═CR^{A}R^{B}, —CR^{C}═CR^{A}R^{B}, —CR^{C}═CHR^{A}, —CR^{C}═CR^{B}, —CCR^{A}, —SH, —SOₖR^{A} (k is 0, 1 or 2) and —NH—C(═NH)—NH₂. Each of R^{A}—R^{D} may independently an aliphatic, substituted aliphatic, benzyl, substituted benzyl, aryl or substituted aryl group, for example, an alkyl, benzylic or aryl group. Further, —NR^{A}R^{D}, taken together, may form a substituted or unsubstituted non-aromatic heterocyclic group. A non-aromatic heterocyclic group, benzylic group or aryl group may also have an aliphatic or substituted aliphatic group as a substituent. A substituted aliphatic group may also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group may have more than one substituent.

Suitable substituents for heteroaryl ring nitrogen atoms having three covalent bonds to other heteroaryl ring atoms may include —OH and C₁ to C₁₀ alkoxy. Substituted heteroaryl ring nitrogen atoms that have three covalent bonds to other heteroaryl ring atoms are positively charged, which may be balanced by counteranions such as chloride, bromide, formate, acetate and the like. Examples of other suitable counteranions may include counteranions found in the described pharmacologically acceptable salts.

Suitable substituents for heteroaryl ring nitrogen atoms having two covalent bonds to other heteroaryl ring atoms include alkyl, substituted alkyl (including haloalkyl), phenyl, substituted phenyl, —S(O)₂-(alkyl), —S(O)₂—NH(alkyl), —S(O)₂—NH(alkyl)₂, and the like.

Also included are pharmaceutically acceptable salts of the compounds described herein. Compounds disclosed herein that possess a sufficiently basic functional group may react with any of a number of organic or inorganic acids to form a salt. Likewise, compounds disclosed herein that possess a sufficiently acidic functional group may react with any of a number of organic or inorganic bases to form a salt. Acids commonly employed to form acid addition salts from compounds with basic groups may include inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts may include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like. Base addition salts include those derived from inorganic bases, such as ammonium or alkali or alkaline earth metal hydroxides, carbonates, bicarbonates, and the like. Such bases useful in preparing the salts of the described compounds may include sodium hydroxide, potassium hydroxide, ammonium hydroxide, potassium carbonate, and the like.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modem Legal Usage 624 (2d. Ed. 1995). Also, to the extent that the terms “in” or “into” are used in the specification or the claims, it is intended to additionally mean “on” or “onto.” To the extent that the term “selectively” is used in the specification or the claims, it is intended to refer to a condition of a component wherein a user of the apparatus may activate or deactivate the feature or function of the component as is necessary or desired in use of the apparatus. To the extent that the term “operatively connected” is used in the specification or the claims, it is intended to mean that the identified components are connected in a way to perform a designated function. To the extent that the term “substantially” is used in the specification or the claims, it is intended to mean that the identified components have the relation or qualities indicated with degree of error as would be acceptable in the subject industry.

As used in the specification and the claims, the singular forms “a,” “an,” and “the” include the plural unless the singular is expressly specified. For example, reference to “a compound” may include a mixture of two or more compounds, as well as a single compound.

As used herein, the term “about” in conjunction with a number is intended to include ±10% of the number. In other words, “about 10” may mean from 9 to 11. Where the term “about” is used with respect to a number that is an integer, the term “about” may mean±10% of the number, or ±5, ±4, ±3, ±2, or ±1 of the number.

As used herein, the terms “optional” and “optionally” mean that the subsequently described circumstance may or may not occur, so that the description includes instances where the circumstance occurs and instances where it does not.

In addition, where features or aspects of the disclosure are described in terms of Markush groups, those skilled in the art will recognize that the disclosure is also thereby described in terms of any individual member or subgroup of members of the Markush group. As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, and the like. As a non-limiting example, each range discussed herein can be readily broken down into a lower third, middle third and upper third, and the like. As will also be understood by one skilled in the art all language such as “up to,” “at least,” “greater than,” “less than,” include the number recited and refer to ranges which can be subsequently broken down into sub-ranges as discussed above. Finally, as will be understood by one skilled in the art, a range includes each individual member. For example, a group having 1-3 members refers to groups having 1, 2, or 3 members. Similarly, a group having 1-5 members refers to groups having 1, 2, 3, 4, or 5 members, and so forth. While various aspects and embodiments have been disclosed herein, other aspects and embodiments will be apparent to those skilled in the art.

As stated above, while the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art, having the benefit of the present application. Therefore, the application, in its broader aspects, is not limited to the specific details, illustrative examples shown, or any apparatus referred to. Departures may be made from such details, examples, and apparatuses without departing from the spirit or scope of the general inventive concept.

1. A compound according to formula I: 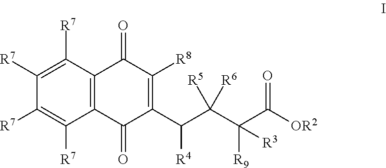
wherein:
R² is —H, C₁-C₁₂ alkyl, C₆-C₁₀ aryl, or C₇-C₁₁ alkylene-aryl optionally substituted with one or more of: R^{a}, and OR^{b},
wherein:
R^{a} and R^{b} are independently —H, C₁-C₆ alkyl, or —CF₃;
R³ is —H, C₁-C₁₂ alkyl, C₆-C₁₀ aryl, or C₇-C₁₁ alkylene-aryl
R⁴, R⁵ and R⁶ are independently —H, C₁-C₆ alkyl, or C₄-C₁₄ aryl or heteroaryl;
each R⁷ and R⁸ are independently —H, halogen, —NO₂, C₁-C₆ alkyl, —N₃, or C₄-C₁₄ aryl or heteroaryl; and
R⁹ is —H or —C(O)OR²;
provided that the compound is not represented by formula 5:

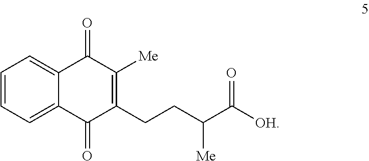 [SMILES: CC1=C(CCC(C)C(=O)O)C(=O)c2ccccc2C1=O]

2. The compound of claim 1, wherein R⁹ is —C(O)OR² and R² is —CH₂CH₃.

3. The compound of claim 1, wherein R⁹ is —C(O)OR² and R² is —H.

4. The compound of claim 1, wherein R⁹ is —H and R² is —H.

5. The compound of claim 1, being represented by one of formulas IB or IC:

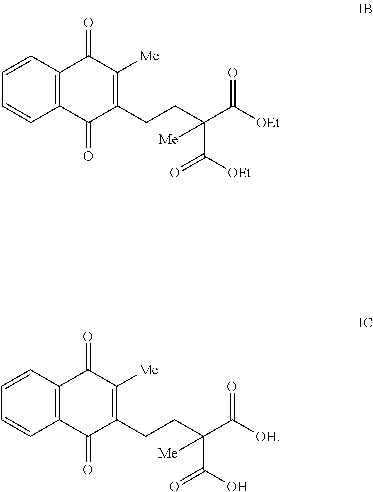 [SMILES: CCOC(=O)C(C)(CCC1=C(C)C(=O)c2ccccc2C1=O)C(=O)OCC ; CC1=C(CCC(C)(C(=O)O)C(=O)O)C(=O)c2ccccc2C1=O]

6. A method for preparing a compound represented by formula I: 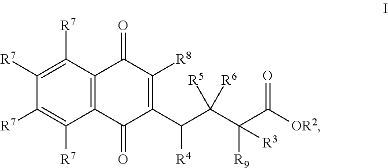
the method comprising:
contacting an acrylate ester compound with a 1,3-diester compound in the presence of a base under a first set of conditions effective to form a triester compound;
contacting the triester compound with a redox reagent under a second set of conditions effective to form a diester acid compound;
contacting the diester acid compound with a naphthoquinone compound in the presence of a radical initiator under a third set of conditions effective to form a naphthoquinone diester compound; and
contacting the naphthoquinone diester compound with a hydrolysis reagent or a saponification reagent to form a solution under a fourth set of conditions effective to form the compound of formula I;
the acrylate ester compound, the 1,3-diester compound, the triester compound, the diester acid compound, the naphthoquinone compound, and the naphthoquinone diester compound being represented by formulas A-F, respectively: 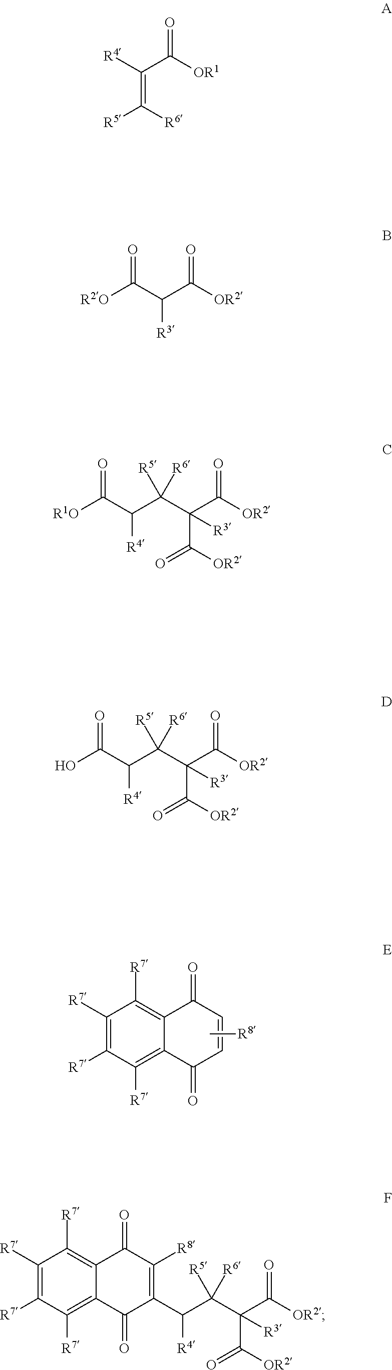
wherein:
R¹ is C₇-C₁₁ alkylene-aryl optionally substituted with one or more of: R^{a}, and OR^{b},
wherein:
R^{a} and R^{b} are independently —H, C₁-C₆ alkyl, or CF₃; and
each R² is independently —H, C₁-C₁₂ alkyl, C₆-C₁₀ aryl, or C₇-C₁₁ alkylene-aryl optionally substituted with one or more of: R^{a}′, and OR^{b}′;
each R²′ is independently C₁-C₁₂ alkyl, C₆-C₁₀ aryl, or C₇-C₁₁ alkylene-aryl optionally substituted with one or more of: R^{a}″, and OR^{b}″,
wherein:
R^{a}″ and R^{b}″ are independently —H, C₁-C₆ alkyl, or CF₃;
R³ and R³′ are —H, C₁-C₁₂ alkyl, C₆-C₁₀ aryl, or C₇-C₁₁ alkylene-aryl;
provided that when:
R²′ is C₇ alkylene-aryl, then
R¹ is C₁₁ alkylene-aryl, or C₇-C₁₁ alkylene-aryl substituted with OR^{b}, wherein R^{b} is C₁-C₆ alkyl; or
R²′ is C₇ alkylene-aryl substituted with one or more of:
R^{a}′, and OR^{b}′ wherein R^{a}′ and R^{b}′ are independently —H, C₁-C₆ alkyl, or CF₃, then
R¹ is C₇-C₁₁ alkylene-aryl;
R⁴, R⁴′, R⁵, R⁵′, R⁶, and R⁶′ are independently —H, C₁-C₆ alkyl, or C₄-C₁₄ aryl or heteroaryl;
each R⁷, each R⁷′, R⁸, and R⁸′ are independently —H, halogen, —NO₂, C₁-C₆ alkyl, —N₃, or C₄-C₁₄ aryl or heteroaryl; and
R⁹ is —H or —C(O)OR².

7. The method of claim 6, wherein R¹ is benzyl and each R² is independently C₁-C₆ alkyl.

8. The method of claim 6, the compound being represented by formula 1A:

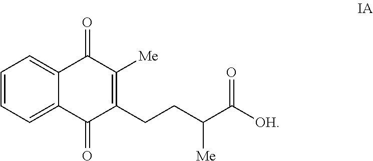 [SMILES: CC1=C(CCC(C)C(=O)O)C(=O)c2ccccc2C1=O]

9. The method of claim 6, the method further comprising removing the base via filtration prior to contacting the triester compound with a redox reagent, the base comprising sodium hydroxide.

10. The method of claim 6, the redox reagent comprising one or more of: a catalyst, H₂, a single-electron transfer oxidant.

11. The method of claim 6, the redox reagent comprising a catalyst and H₂.

12. The method of claim 11, the catalyst comprising one or more of: palladium (Pd), platinum (Pt), rhodium (Rh), ruthenium (Ru), or Nickel (Ni).

13. The method of claim 11, the catalyst being palladium on carbon (Pd/C).

14. The method of claim 6, the radical initiator comprising a peroxydisulfate salt and a silver(I) salt.

15. The method of claim 14, the peroxydisulfate salt being ammonium persulfate.

16. The method of claim 14, the silver salt being silver nitrite.

17. The method of claim 6, comprising the hydrolysis reagent, the hydrolysis reagent comprising a Brønstad acid.

18. The method of claim 6, the fourth set of conditions comprising heating the solution to a temperature of at least 100° C.

19. The method of claim 6, the fourth set of conditions comprising heating the solution for a period of time, and subsequently contacting the solution with a decarboxylation promoter, the decarboxylation promoter being N,N-carbonyldiimidazole.
